(19) United States
(12) Patent Application Publication
(10) Pub. No.: US 2005/0153422 A1
(43) Pub. Date: Jul. 14, 2005

(54) Novel Pseudonocardia sp. RMRC PAH4 and a process for bioconverting compactin into pravastatin using the same

(75) Inventors: Chung-Liang Lin, Chiayi City (TW); Tung-Li Huang, Chiayi City (TW); Jeen-Kuan Chen, Chiayi City (TW); Chi-Sheng Wu, Chiayi City (TW); Bi-Ru Tsau, Taipei Hsien (TW)

Correspondence Address: BROWN & MICHAELS, PC;400 M & T BANK BUILDING, 118 NORTH TIOGA ST, ITHACA, NY, 14850, US

(73) Assignee: to Chinese Petroleum Corporation, Taipei City (TW)

(21) Appl. No.: 11/002,380

(22) Filed: Dec. 2, 2004

(30) Foreign Application Priority Data
Jan. 9, 2004 (TW) 093100506

(51) Int. Cl. C12N 1/21

(52) U.S. Cl. 435/252.3

(57) ABSTRACT

The invention provides a novel microorganism Pseudonocardia sp. RMRC PAH4 characterized in that it is able to degrade high concentration of quinoline by enrichment culture, shows a high tolerance to compactin-sodium and possesses a high hydroxylation activity of converting compactin-sodium to pravastatin-sodium. The invention relates also a process for converting compactin-sodium into pravastatin-sodium by fermenting said novel microorganism Pseudonocardia sp. RMRC PAH4. Pravastain-sodium is a potent cholesterol-lowering agent used in treatment for hypercholesterolemia.

### BACKGROUND OF THE INVENTION

1. Field of the Invention

The invention relates to a novel microorganism Pseudonocardia sp. RMRC PAH4 and a process for converting compactin-sodium into pravastatin-sodium by fermenting said novel microorganism Pseudonocardia sp. RMRC PAH4.

2. Description of the Prior Art

Cerebravascular disease, cardiac disease and complications thereof are attributed to atherosclerosis in which dyslipidemia is the uppermost exacerbating factor. Among them, hyperlipidemia is known as an excessive high level of main lipids, i.e., cholesterol and triglyceride (the neutral lipid), in the blood. Cholesterol is synthesized from basic acetyl-CoA unit through more than 20 steps, in which the bottleneck reaction resides on the step of conversion of HMG-CoA into mevalonic acid catalyzed by HMG-CoA reductase. A “Statins era” has been opened since Mevacor (Lovastatin) was commercially available in 1987. Statins commonly used are Mevacor, Zocor (Simvastatin), Pravachol (Pravastatin), Lipitor (Atorvastatin) and the like. The mechanism for the action of the essential component of these drugs consists of inhibiting the activity of HMG-CoA reductase and hence lowering the yield of cholesterol.

Pravastatin, due to its one hydroxy group, is a highly hydrophilic compound, can act selectively on the main cholesterol-synthesizing organs, i.e., liver and small intestine, blocks the biosynthesis of cholesterol, lowers the level of cholesterol, enhances the activity of low density lipoprotein, increases the uptake of low density lipoprotein from blood into liver and hence lowers the level of low density lipoprotein in the serum, can decrease rapidly and potently serum cholesterol, as well as improves serum lipid. In addition, due to its organ selectivity, Pravastatin acts weakly on organs, such as hormone-producing organ, other than liver and small intestine, and therefore, it has little side effect. In spite of the most complex process for preparing Pravastatin, it is one of the most efficient chloesterol-lowering agents known heretofore. Pravastatin not only exhibits excellent therapeutic effect on lowering low-density lipoprotein-cholesterol, but also has been found clinically that, in terms of anti-atherosclerosis or the therapeutic effect for reducing cadiovascular disease, it is far more effective than other cholesterol-lowering drugs. Additionally, it is safe for long-term usage, does not increase the incidence of tumor, is convenient for use as well as has little side effect. Consequently, it has a place in the market. On the other hand, new therapeutic effect of Pravastatin is consistently found out, such as, for example, as reported in Archives of Neurology 2000; 57: 1439-1443 that the incidence of currently popular senile dementia can be decreased by Pravastatin up to 73%. Further, it has been reported that Pravastatin could reduce the incidence of stroke up to 22% (Byington R P, Davis B R, Plehn J F, White H D, Baker J, Cobbe S M, Shepherd J. Reduction of stroke events with pravastatin: the Prospective Pravastatin Pooling (PPP) Project. Circulation. 2001 Jan. 23; 103(3):387-92.). Although these preliminary observations could not be considered as medical evidences, the medical practitioner will increase more or less the amount or possibility of using Pravastatin under these hints in their prescribe accordingly. Furthermore, under the circumstance of increasingly improved pharmaceutical formulation and technology, Pravastatin may be switched from prescription into officinal, suggesting a very considerable market scale. Anyhow, the future of Pravastatin is greatly expected.

Two-stage process for producing Pravastatin refers to synthesize compactin firstly by fermentation, and then hydroxylation of compactin into Pravastatin using enzyme groups thus produced by the microorganism. Bacterial strains that can convert biologically compactin into Pravastatin include, for example, Streptomyces roseochromogenu NRRL-1233, Streptomyces roseochromogenus IFO-3363, Streptomyces roseochromogenus IFO-3411 (U.S. Pat. No. 4,346,227), Streptomyces carbophilus SANK-62585 (Ferm BP-1145; U.S. Pat. No. 5,179,013), and Streptomyces halstedii (JP No. 4-349034). However, the original stain of these microorganisms does not have high tolerance against compactin, leading to a low yield of Pravastatin. Recently, a number of patents disclosed strains that exhibited very high tolerance against compactin and had conversion rate of more than 50%. These strains were, for example, Streptomyces exfoliates yj-118 (U.S. Pat. No. 3,306,629), Micromonospora sp. (WO Pat. No. 0103647), Actinomadura sp. ATCC 55678 (U.S. Pat. No. 6,274,360).

Among them, Streptomyces carbophilus) can bioconvert compactin into Pravastatin through a hydroxylation reaction which can not accomplished by only one enzyme, but by a reaction cascade consisting of reactions catalyzed by Cytochrome P450, reductase, NADH or NADPH regeneration system. Accordingly, though microorganisms that can convert compactin into Pravastatin are present commonly in the nature, there is rare one that has economical value in terms of compactin tolerance and conversion efficiency.

### SUMMARY OF THE INVENTION

Accordingly, one object of the invention is to provide a novel Pseudonocardia sp. RMRC PAH4 characterized in that this strain can degrade quinoline, has a very high tolerance against compactin sodium, and it can bio-convert efficiently compactin sodium into pravastatin sodium.

Another object of the invention is to provide a process for converting compactin sodium into Pravastatin sodium characterized in that the novel above-described strain, Pseudonocardia sp.RMRC PAH4, according to the invention is used in the process to bio-convert compactin sodium into Pravastatin sodium.

### BRIEF DESCRIPTION OF THE DRAWINGS

The drawings disclose an illustrative embodiment of the present invention which serves to exemplify the various advantages and objects hereof, and are as follows:

FIG. 1 shows the electromicrograph of aerobic mycelium and spore chain of Pseudonocardia sp. RMRC PAH4; and

FIG. 2 shows the vegetative mycelium of Pseudonocardia sp. RMRC PAH4.

### DETAILED DESCRIPTION OF THE PREFERRED EMBODIMENT

The novel strain, Pseudonocardia sp.RMRC PAH4, according to the invention has a good ability of degrading polycyclic aromatic hydrocarbon, i.e., quinoline, and also a high tolerance to compactin sodium, so that it can be used to convert effectively compactin sodium into Pravastatin sodium through incubation by fermentation.

Pravastatin can be used as raw material of cholesterol-lowering agent. Heretofore, no patent or literature reports use of this strain for the production of Pravastatin.

The novel Pseudonocardia sp. RMRC PAH4 strain according to the invention will be described more detailed hereinafter.

1. Culture Screening

In the culture library of CHINESE PETROLEUM CORPORATION, there are a number of polycyclic aromatic hydrocarbon-converting strains isolated from contaminated soil. These strains can degrade independently or in combination quinoline, benz[a]anthracene or naphthalene. These strains were cultured individually in YM liquid medium consisting of yeast extract 0.3%, maltoextract 0.3%, peptone 0.5%, glucose 1%, pH 6.5, at 200 rpm, under 28° C. for 48 hours. Then, 100-1,000 μg/ml of compactin sodium was added and continued the incubation for 24-72 hours. The utilization rate of compactin sodium and the conversion rate of Pravastatin sodium were determined quantitatively by HPLC. The results indicated that, under these conditions, there were seven strains that could convert compactin into Pravastatin. Among them, Pseudonocardia sp. RMRC PAH4 had the highest tolerance against compactin sodium, and could convert efficiently compactin sodium into Pravastatin sodium. This strain belonged to a strain that could decompose quinoline.

1. Strain Identification

Pseudonocardia sp. RMRC PAH4 had been strain identified by Food Industry Research and Development Institute (FIRDI) based on following analysis of cellular chemical components, morphological characteristics of the strain and comparison of the physiological and biochemical features of its mycelium. As the result, this strain was identified as Pseudonocardia alni, and was denominated as Pseudonocardia sp. RMRC PAH4. This strain was deposited in FIRDI (Food Industry Research and Development Institute, 331 Shin-Pin Road Hsinchu, 300 Taiwan, R.O.C.) under the accession number as BCRC 910209.

This strain was also deposited in the DSMZ (Deutsche Sammlung von Mikroorganismen und Zelkulturen GmbH) on Nov. 26, 2004. The DSMZ accession number is DSM 16946.

(i) Analysis of Cellular Chemical Components

The cell wall amino acid and sugar components in the whole cell were meso-DAP, and galactose, arabinose, glucose and ribose, respectively. According to the classification of Lechevalier et al., it belongs to Chemotype III A. It does not contain substantially mycolic acid. Its major mesoquinone type is MK-8(H₄), and contains large amount of iso-C16 : 0,anteiso-C15 : 0 as well as minor amount of methyl fatty acid, i.e., 10methyl-C16: 0,C17 : 0,C18 : 0.

(ii) Morphological Characteristics of the Strain

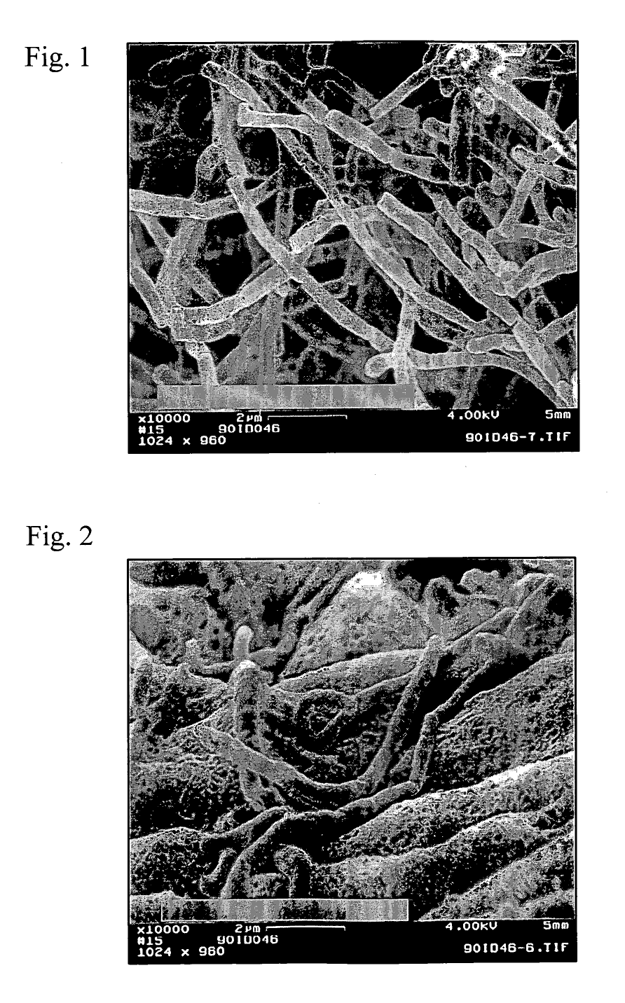

The vegetative mycelium of the strain on the culture medium is yellowish-brown or yellowish white. Its aerobic mycelium is milk white. It does not produce soluble pigment or melanin (Table 1). It produces straight spore chain on the aerobic mycelium. The surface of the spore is smooth. Its vegetative mycelium has a number of branches and may fragment ate (FIG. 1 and 2).

(iii) Utilization of Carbohydrates and Substances by the Strain is Shown in Table 2.

(iv) Based on the Above-described Results and Comparison with Reference to Bergey's Manual of Systematic Bacteriology, this Strain was Identified as Pseudonocardia alni.

3. The Tolerance of Pseudonocardia sp. RMRC PAH4 Against Compactin Sodium

(1) Culture Incubation

The strain was inoculated in the culture medium consisting of casein hydrolysate 0.05-0.2%; yeast extract 0.05-0.2%; soluble starch 0.5-2.0%; KH₂PO₄ 0.01-0.08%; MgSO₄.7H₂O 0.05-0.2%; Pravastatin sodium 0.005-0.01%; and Bacto agar 2.0%, pH 7.0, and incubated under 28° C. for 7-20 days.

(2) Fermentation on Shaker

The above inoculum culture was incubated in compactin sodium-containing YMG liquid medium consisting of yeast extract 0.1-1.0%; malto extract 0.1-1.0%; peptone 0.1-1.0%; glucose 0.5-2.0%; cotton seed extract (Pharmamedia) 0.5-0.5%; KH₂PO₄ 0.1-0.5%; Na₂HPO₄ 0.3-0.7%; MgSO₄.7H₂O 0.01-0.05%; FeSO₄.7H₂0 0.001-0.01%; MnSO₄ . H₂O 0.001-0.01%; CaCl₂ 0.001-0.01%; compactin sodium 0.002-0.01%; pH 6.5, on a shaker, at 220 rpm, under 28° C.

(3) Tolerance of Pseudonocardia sp.RMRC PAH4 Against Compactin Sodium

After incubating on shaker as described above for 48 hours, 300-3,000 μg/ml of compactin sodium as added and continued the incubation under same conditions. Utilization rate of compactin sodium and conversion rate of Pravastatin sodium were analyzed by HPLC at an interval of 24 hours. The result indicated that as compactin sodium was added in an amount more than 2,500μg/ml, the growth of the bacteria will become considerably slowly, and the conversion rate of Pravastatin could not exceed 30%.

5. Ability of Pseudonocardia sp. RMRC PAH4 to Convert Compactin Sodium into Pravastatin Sodium

To a YMG liquid medium containing 0.002-0.01% compactin sodium was inoculated 3-10% of the cultured strain and incubated at 220 rpm, under 28° C. for 48 hours. Then, 500-1,500 μg/ml of compactin sodium was added and continued incubation under same conditions. When the pH of the culture medium exceeded 7.0, 0.1-0.8% of glucose, 0.05-0.5% of yeast extract, and 0.05-0.5% of cottonseed extract (Pharmamedia) were added. For every interval of 24 hours, utilization rate of compactin sodium and conversion rate of Pravastatin sodium were analyzed by HPLC. Results indicated that, after incubating for 36-72 hours, the utilization rate of compactin sodium was more than 90%, while the conversion rate of Pravastatin was approximately 50-68%.

The invention provides also a process for bioconvert compactin sodium into Pravastatin sodium by using the above-described novel Pseudonocardia sp. RMRC PAH4, wherein the fermentation process mentioned above was used to convert efficiently compactin sodium into Pravastatin sodium with a conversion rate of 50-68%.

The invention will be illustrated further by following non-limiting example.

### EXAMPLES

### Example 1

The culture strain was inoculated in the culture medium consisting of casein hydrolysate 0.1%; yeast extract 0.1%; soluble starch 1%; KH₂PO₄ 0.05%; MgSO₄.7H₂0 0.1%; Pravastatin sodium 0.005; and Bacto agar 2.0%, pH 7.0, and incubated under 28° C. for 7-20 days.

To a 500-ml Erlenmeyer flask containing 60 ml of YMG liquid medium consisting of the strain culture was inoculated in the culture medium consisting of yeast extract 0.4%; maltoextract 0.35%; soluble starch 0.5-2.0%; peptone 0.6%; glucose 1.0%; cottonseed extract (Pharmamedia) 0.2%, KH₂PO₄ 0.1%;Na₂HPO₄ 0.4%;MgSO₄.7H₂O 0.02%;FeSO₄.7H₂O 0.005%;MnSO₄.H₂O 0.002%;CaCl₂ 0.002%; compactin sodium 0.005%; pH 6.5, was inoculated 3-10% of the culture strain and incubated on a shaker at 220 rpm, under 28° C. for 48 hours. 500 μg/ml of compactin sodium was added and continued incubation under same conditions. For every interval of 12 hours, utilization rate of compactin sodium and conversion rate of Pravastatin sodium were analyzed by HPLC. Results were shown in Table I.

Condition for HPLC analysis was as follow:
Column: C1 8,4.6×250 mm
Detector: UV 238 nm
Flow rate: 0.8 ml/min
Mobile phase:methanol:triethylamine (TEA):acetic acid:H₂O=70:0.1:0.1:30

Oven temperature: 35° C.

TABLE I
The ability of Pseudonocardia sp. RMRC PAH4
to bioconvert compactin into Pravastatin*

| # of hours | | | Bioconversion rate |
|---|---|---|---|
| after addition | compactin | Pravastatin | vs added compactin |
| of compactin | (μg/ml) | (μg/ml) | (%) |
| 12 | 82 | 156 | 31.2 |
| 24 | 16 | 262 | 52.4 |
| 36 | 0 | 273 | 54.6 |
| 48 | 0 | 281 | 56.2 |
| 60 | 0 | 279 | 55.8 |
| 72 | 0 | 281 | 56.2 |

*500 μg/ml compactin sodium was added after growing for 2 days.

### Example 2

This example was performed under the same conditions as in example 1 except addition of 1,000 μg/ml of compactin sodium. The result was shown in Table II.

TABLE II
The bioconverion ability of Pseudonocardia sp. RMRC PAH4
under incubation condition of high concentration of compactin*

| # of hours | | | Bioconversion |
|---|---|---|---|
| after addition | Compactin | Pravastatin | rate vs added |
| of compactin | (μg/ml) | (μg/ml) | compactin (%) |
| 12 | 402 | 226 | 22.6 |
| 24 | 180 | 314 | 31.4 |
| 36 | 78 | 412 | 41.2 |
| 48 | 44 | 488 | 48.8 |
| 60 | 28 | 520 | 52.0 |
| 72 | 18 | 518 | 51.8 |

*1,000 μg/ml compactin sodium was added after growing for 2 days.

### Example 3

This example was performed under condition as example 1. To YMG liquid production medium containing 0.005% compactin sodium was inoculated 5% bacterial inoculum, incubated at 220 rpm, under 28° C. for 48 hours. Thereafter, 1,000 μg/ml compactin sodium was added and continued incubation under same condition. At an interval of 48 hours, to the medium was added 0.1-0.8% of glucose, 0.05-0.5% of yeast extract, and 0.05-0.5% cottonseed extract (Pharmamedia). For every interval of 24 hours, utilization rate of compactin sodium and conversion rate of Pravastatin sodium was determined by HPLC. The result was shown in Table III.

TABLE III
Ability of Pseudonocardia sp. RMRC PAH4 to synthesize Pravastatin*

| # of hours after | | | Bioconversion |
|---|---|---|---|
| addition of | compactin | Pravastatin | rate vs added |
| compactin | (μg/ml) | (μg/ml) | compactin (%) |
| 1 | 182 | 300 | 30.0 |
| 2** | 56 | 466 | 46.6 |
| 3 | 24 | 604 | 60.4 |
| 4** | 18 | 612 | 61.2 |
| 5 | 0 | 678 | 67.8 |
| 6 | 0 | 680 | 68.0 |

*1,000 μg/ml compactin was added after growing for 2 days.
**Glucose, yeast extract and cottonseed extract (Pharmamedia) had been added.

### Example 4

This example was performed under conditions as example 3, except that 1,000 μg/ml of compactin sodium was added for every 12-48 hours and continued incubation under same conditions. When pH of the medium was higher than 7.0, 0.1-0.8% of glucose, 0.05-0.5% of yeast extract and 0.05-0.5% of cottonseed extract (Pharmamedia) were added. After 9 days, the total amount of compactin sodium added was 7,000 μg/ml, the utilization rate of compactin sodium was about 76%, and the conversion rate of Pravastatin was about 53%.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

TABLE 1
Cultural characteristics of Pseudonocardia alni on ISP media

| Characteristics | | | | | Soluble |
|---|---|---|---|---|---|
| medium | Growth | Substrate mycelia | Aerial mycelia | Sporulation | pigment |
| Yeast extract-malt | Well | Strong yellowish | Yellowish white | Well | None |
| extract agar(ISP 2 | | brown | | | |
| medium) | | | | | |
| Oatmeal agar(ISP 3 | Well | Pale yellow | Yellowish white | Well | None |
| medium) | | | | | |
| Inorganic salts starch | Moderate | Yellowish white | Yellowish white | Moderate | None |
| agar (ISP 4 medium) | | | | | |
| Glycerol asparagines | Moderate | Light yellowish | Yellowish white | Poor | None |
| agar (ISP 5 medium) | | brown | | | |

TABLE 2
Physiological characteristics of Pseudonocardia alni.

| Character | |
|---|---|
| Decomposition of: | |
| L-tyrosine | +* |
| Casein | + |
| Xanthine | + |
| Hypoxanthine | + |
| Esculin | + |
| Urease production | − |
| Nitrase production | − |
| Lysozyme resistance | − |
| Melanin production | − |
| Gelatin liquidation | − |
| Utilization of the following compounds | |
| As sole carbon and energy sources: | |
| meso-Inositol | +/− |
| D-Mannitol | + |
| L-Rhamnose | + |
| L-Arabinose | +/− |
| Glucose | + |
| Xylose | + |
| Sucrose | +/− |
| Fructose | + |
| Raffinose | +/− |
| Salicin | + |
| Adonitol | + |
| Proline | + |
| Cellobiose | + |
| Galactose | + |
| Gluconate | + |
| Uracil | − |
| Lactose | +/− |
| Sorbitol | +/− |
| Maltose | + |

*+: positve reaction,
−: negative reaction,
+/−: weak reaction

1. A novel strain Pseudonocardia sp. RMRC PAH4, characterized in that said strain can degrade quinoline, has a tolerance against compactin sodium of more than 500μg/ml, and can convert efficiently compactin sodium into Pravastatin sodium by biological means, wherein said Pseudonocardia sp. RMRC PAH4 exhibits following microbiological characteristic:
(i) analysis of cellular chemical components
the cell wall amino acid and sugar components in the whole cell being meso-DAP, and galactose, arabinose, glucose and ribose, respectively; according to the classification of Lechevalier et al., it belonging to Chemotype III A; containing substantially no mycolic acid; major mesoquinone type being MK-8(H₄), and containing large amount of iso-C16 : 0,anteiso-C15 : 0 as well as minor amount of methyl fatty acid, i.e., 10 methyl-C16: 0,C17 : 0,C18 : 0;
(ii) morphological characteristics of the strain
the vegetative mycelium of the strain on the culture medium being yellowish-brown or yellowish white; its aerobic mycelium being milk white; producing no soluble pigment or melanin (Table 1); producing straight spore chain on the aerobic mycelium; the surface of the spore being smooth; its vegetative mycelium having a number of branches and fragments (FIG. 1 and 2);
(iii) utilization of carbohydrates and substances by the strain as shown in Table 2; and
(iv) based on the above-described results and comparison with reference to Bergey's Manual of Systematic Bacteriology, this strain being identified as Pseudonocardia alni.

2. A Pseudonocardia sp. RMRC PAH4 as in claim 1,wherein said Pseudonocardia sp. RMRC PAH4 is deposited in Food Industry Research and Development Institute (FIRDI) under accession No.: BCRC910209.

3. A process for convert compactin sodium into Pravastatin sodium, characterized in that the Pseudonocardia sp. RMRC PAH4 as defined in claim 1 is used in a fermentation process to convert compactin sodium into Pravastatin sodium, wherein said fermentation process consists of inoculating the bacterial in the culture medium consisting of casein hydrolysate 0.05-0.2%; yeast extract 0.05-0.2%; soluble starch 0.5-2.0%; KH₂PO₄ 0.01-0.08%; MgSO₄.7H₂0 0.05-0.2%; Pravastatin sodium 0.005-0.01%; and Bacto agar 2.0%, pH 7.0, and incubating under 28° C. for 7-20 days; thereafter, to a 500-ml Erlenmeyer flask containing YMG liquid producing medium consisting of yeast extract 0.1-1.0%; maltoextract 0.1-1.0%%; soluble starch 0.5-2.0%; peptone 0.1-1.0%%; glucose 0.5-2.0%%; cottonseed extract 0.5-0.5%; KH₂PO₄ 0.1-0.5%; Na₂HPO₄ 0.3-0.7%; MgSO₄.7H₂O 0.01-0.05%; FeSO₄.7H₂0 0.001-0.01%; MnSO₄ . H₂O 0.00 CaCl₂ 0.001-0.01%; compactin sodium 0.002-0.01%; pH 6.5, inoculating 3-10% of the culture strain and incubating on a shaker at 220 rpm, under 28° C. for 40-60 hours, and then adding 500 μg/ml of compactin sodium, incubating under same conditions.

4. A process as in claim 3, wherein said Pseudonocardia sp. RMRC PAH4 is in Food Industry Research and Development Institute (FIRDI) under accession No.: BCRC910209.

5. A process as in claim 3, wherein said amount of compactin sodium added in YMG liquid producing medium is 300-3,000 μg/ml.
